(19) United States
(12) Patent Application Publication
(10) Pub. No.: US 2018/0322948 A1
(43) Pub. Date: Nov. 8, 2018

(54) INFUSION PUSH ALERTS

(71) Applicant: Smiths Medical ASD, Inc., Plymouth, MN (US)

(72) Inventors: James B. DROST, Woodbury, MN (US); James SURINE, Oakdale, MN (US); Eric WILKOWSKE, North Oaks, MN (US)

(73) Assignee: Smiths Medical ASD, Inc., Plymouth, MN (US)

(21) Appl. No.: 15/773,483

(22) PCT Filed: Oct. 14, 2016
(86) PCT No.: PCT/US2016/056952

§ 371 Date: May 3, 2018

Related U.S. Application Data
(60) Provisional application No. 62/252,925, filed on Nov. 9, 2015.

(51) Int. Cl. G16H 20/17; G16H 10/65; G16H 40/67; G16H 80/00

(52) U.S. Cl. CPC G16H 20/17; G16H 80/00; G16H 40/67; G16H 10/65

(57) ABSTRACT

Embodiments include methods, apparatuses and systems related to providing push alerts via infusion pumps. In one embodiment, a method includes receiving an infusion order for a patient in a computerized physician order entry system including a patient identifier. The method further includes obtaining verification of the infusion order from a pharmacy and sending an infusate requested in the infusion order from the pharmacy to a patient location. The method also includes updating an electronic medication administration record (EMAR) system with an approval to implement the infusion order. The method includes providing a notification from an alert component in one or more devices associated with the patient identifier of updates to the EMAR system, without a practitioner initiated inquiry, and at least one of the one or more devices is an infusion pump at the patient location.

### TECHNICAL FIELD

Embodiments relate generally to communication and control devices, systems and methods for medical devices and, more particularly, to devices, systems, and methods providing push alerts to infusion pumps and related devices.

### BACKGROUND

In the medical arts, medical devices such as infusion pumps have been useful for managing the delivery and dispensation of prescribed amounts or doses of drugs, fluids, fluid-like substances, or medicaments (hereinafter, collectively, “infusates”) to patients. Infusion pumps can provide some significant advantages over manual infusion techniques, by accurately delivering and dispensing infusates over an extended period of time. Infusion pumps are particularly useful for treating diseases and disorders that require regular pharmacological intervention, including cancer, diabetes, and vascular, neurological, and metabolic disorders. They also enhance the ability of healthcare providers to deliver anesthesia and manage pain. Infusion pumps are used in various settings, including hospitals, nursing homes, and other short-term and long-term medical facilities, as well as in residential care settings. Infusion pumps can include various constructions, modes of operation, and types. Generally, infusion pumps include portable or ambulatory pumps, large volume pumps (LVPs), patient-controlled analgesia (PCA) pumps, peristaltic pumps, elastomeric pumps, syringe pumps, enteral pumps, and insulin pumps. Depending upon their specific designs and intended uses, infusion pumps can be used to administer infusates through various delivery methods, including intravenously, intraperitoneally, intra-arterially, subcutaneously, neuraxially, and specifically into an intraoperative site, epidural space, and subarachnoid space.

In hospitals, clinics, and other medical environments, patient safety continues to be a paramount concern along with improved workflow efficiencies. This has been especially true when dealing with vulnerable patients and situations in which potent infusates capable of causing significant physiological or chemical effects are being administered. Accordingly, medical practitioners strive to ensure that patients receive safe and appropriate medical care including appropriate infusions of infusates.

Infusion pumps may be controlled locally via the programming of each individual pump. For example, a medical practitioner can configure an infusion pump to execute a delivery profile that corresponds to a patient's treatment needs, or a patient can configure an infusion pump according to their individual requirements within pre-defined limits without the involvement of a physician. Alternatively, infusion pumps may be controlled via other techniques such as by, for example, a network server that communicates with the pumps. Hospital information systems (HIS) and electronic medical record (EMR) systems may, in some circumstances, provide such network functionality.

Practitioners are increasingly responsible for attending to numerous pumps, devices, and medical care activities associated with one or several patients. For practitioners, efficiently tracking and performing tasks during their day is increasingly difficult in many respects, as treatments, medications and courses of care can be changed quickly within the care facility or environment and its associated electronic system or systems.

Accordingly, there is a need for devices, systems, and methods for alerting practitioners to changes required for infusion pumps. An infusion pump or related system providing alerts that are more efficient and effective is desired. Various new pumps, systems, methods and other arrangements could be decidedly advantageous in improving patient safety, workflow efficiency, programming, control, and operational parameters, etc., for infusion pumps and other coordinated medical devices.

### SUMMARY

Embodiments described or otherwise contemplated herein substantially provide the aforementioned advantages of providing or improving patient safety, workflow efficiency, programming, control, and operational parameters, among possible other advantages. Improvements in methods, systems, and apparatuses regarding notifications and alerts related to infusion pumps and treatments in medical environments are described.

One embodiment relates to a method of providing push alerts of infusion pump orders. The method includes receiving an infusion order for a patient in a computerized physician order entry system including a patient identifier and obtaining verification of the infusion order from a pharmacy. The method further includes sending data concerning an infusate, requested in the infusion order from the pharmacy, to a patient location and also includes updating an electronic medication administration record (EMAR) system with an approval to implement the infusion order. The method further includes providing a notification from an alert component in one or more devices associated with the patient identifier of updates to the EMAR system, without a practitioner initiated inquiry. Further, at least one of the one or more devices is an infusion pump at the patient location.

Another embodiment includes a method of providing push alerts of infusion pump orders. The method includes receiving manual programming by a practitioner of a first infusion pump order, including a patient identifier of a patient, associating a plurality of devices with the patient identifier, and receiving a second infusion pump order associated with the patient identifier. The method also includes sending a message to the plurality of devices associated with the patient identifier regarding the second infusion pump order. The method further includes providing one or more audio, visual, or tactile alert notifications regarding the second infusion pump order from an alert component of each of the plurality of devices associated with the patient identifier without requiring a practitioner action.

Another embodiment relates to an infusion pump with push alert notification. The infusion pump includes a pump housing, a pumping mechanism coupled to the pump housing that selectively delivers an infusate to a patient, and a pump control system including a processor and a memory programmable to control operation of the pumping mechanism. The infusion pump also includes an alert component, coupled with the pump housing, with audio, visual, or tactile alerts. Further, the infusion pump has an alert control engine, in operable communication with the pump control system. The alert control engine includes programming that associates a patient identifier for the patient with the infusion pump, receives an alert message from a local subnet related to an infusion order associated with the patient identifier, and instructs the alert component to provide an alert notification to a practitioner related to the infusion order without requiring an inquiry by the practitioner.

A further embodiment relates to a system of push alert notification for infusion pumps. The system includes a group of selected devices associated with a local subnet and an infusion pump. The infusion pump includes a pump housing, a pumping mechanism coupled to the pump housing that selectively delivers an infusate to a patient, and a pump control system including a processor and a memory programmable to control operation of the pumping mechanism. The infusion pump further includes an alert control engine in operable communication with the pump control system. The alert control engine includes programming that associates a patient identifier, for sending future notifications, with the group of selected devices following receipt of programming for a first order for the infusion pump for the patient. The alert control engine receives a second order for the infusion pump, generates an alert message related to the second order, and sends the alert message to the group of selected devices. Each of the group of selected devices and the infusion pump each contain an alert component that provides an audio alert, a visual alert, or a tactile alert when the alert message is sent for practitioner notification.

The foregoing summary is not necessarily intended to describe each illustrated embodiment or every implementation of the subject matter hereof. The figures and the detailed description that follow more particularly exemplify the embodiments.

### BRIEF DESCRIPTION OF THE DRAWINGS

Subject matter hereof may be more completely understood in consideration of the following detailed description of various embodiments of the subject matter in connection with the accompanying drawings, in which:

FIGS. 1A-C show various infusion pumps configured with push alert notification features and use with push alert systems, according to various embodiments.

FIG. 2 is a block diagram of various elements of an infusion pump system with push alert notification, according to various embodiments.

FIG. 3 is an example of a mounting rack providing a local subnet including a plurality of infusion pumps coupled to the mounting rack configured for push alerts, according to an embodiment.

FIG. 4 is a diagram of a mounting rack arrangement providing a local subnet including a plurality of medical devices configured for push alerts, according to an embodiment.

FIG. 5 is a diagram of a method for completing infusion orders for infusion pumps including “pull” or “push” notification of information to or by a practitioner, according to an embodiment.

FIG. 6 is a diagram describing an infusion pump push alert method, according to an embodiment.

FIG. 7 is a diagram depicting patient identifier association for programmed infusions in a subnet, according to an embodiment.

FIG. 8 is a block diagram of various elements of an infusion pump system with push alert notification, according to an embodiment.

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION

Medical devices and systems including one or more programmed infusion pumps and/or other medical devices are increasingly complex and potentially prone to errors. The need for and advantages of a simplified and less error-prone alert system for such medical devices and systems has been recognized by applicants. The following disclosure relates therefore to technology and methods of such advanced notification systems.

One area of medical care which applicants have identified for improvement relates to efficiently providing alerts to practitioners or other users of infusion pumps when new orders are placed, when orders are modified, or when activities have occurred which require attention.

Throughout this application the term “practitioner” is intended to refer to any nurse, physician, medical practitioner, hospital staff or other medical care worker responsible for operating medical equipment or devices.

Presently, in many medical care facilities, when a patient infusion order changes or a new infusion order is entered, a practitioner might not immediately know of this change. The practitioner typically must log into an electronic system to check their work list to be notified of such changes. This delay in information exchange can correspondingly cause delays in patient therapy and less than optimal efficiency and care. Accordingly, the described push alert system, where practitioners are efficiently notified of infusion pump orders and order modifications, is believed to be highly desirable in hospitals and other medical environments.

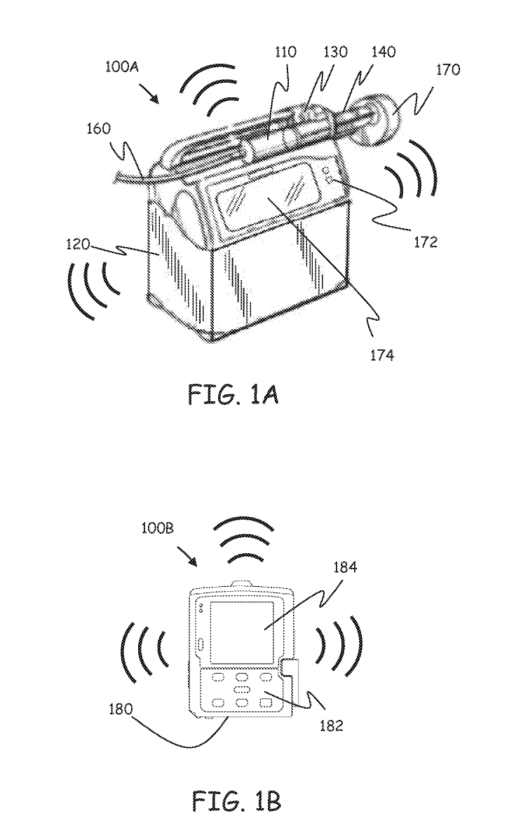

FIGS. 1A-B show various pumps 100A-B as examples of medical devices that can be used in Inform/Advise/Control systems and which can be configured with various alert notification capabilities and into push alert notification systems in certain embodiments as described herein. Pumps 100A-B also represent examples of various infusion pumps that can be used in push alert systems that utilize local subnets as described herein.

Infusion pump 100A shown in FIG. 1A is a syringe-type pump that can be used to deliver a wide range of infusate therapies and treatments. Infusion pump 100A typically includes a pharmaceutical container or syringe 110, which is supported on and secured to housing 120 by clamp 130, respectively. In embodiments, syringe 110 can be separately supplied from pump 100A. In other embodiments, syringe 110 is an integrated component of pump 100A. Syringe 110 typically includes a plunger 140 that forces fluid outwardly from syringe 110 via infusion line or tubing 160 that is connected to a patient. A motor and lead screw arrangement internal to housing 120 of pump 100A cooperatively actuates a pusher or plunger driver mechanism 170, to move plunger 140. In embodiments, a sensor can monitor force and/or position of plunger 140 in syringe 110 according to system specifications. Also, depicted on pump 100A is at least one user interface 172 for operator input and/or display 174. In various embodiments, the display 174 can also be combined with or serve as part of a user interface 172 as a touch screen or touchless interface, for example.

Infusion pump 100B shown in FIG. 1B is an example of an ambulatory infusion pump that can be used to deliver a wide range of infusate therapies and treatments. Such an ambulatory pump can typically be comfortably worn by way of belts, straps, clips or other simple fastening means or be provided in ambulatory pole-mounted arrangements within hospitals and other medical care facilities.

Infusion pump 100B generally includes a peristaltic type infusion pump mechanism that controls a flow of infusate from a reservoir (not shown in FIG. 1B) coupled to pump 100B through a conduit from the reservoir which matingly passes along bottom surface 180 of pump 100B. The reservoir can, for example, comprise a cassette (not shown in FIG. 1B) that is attached to the bottom of pump 100B at surface 180, or an IV bag or other fluid source that is similarly connected to pump 100B via an adapter plate (not shown in FIG. 1B) at surface 180. Specifically, pump 100B typically uses valves and an expulsor located on bottom surface 180 to selectively squeeze a tube of fluid (not shown in FIG. 1B) connected to the reservoir to effect the movement of the fluid supplied by the reservoir through the tube and to a patient in peristaltic pumping fashion. Pump 100B also has at least one user interface 182 for operator input and/or display 184. In various embodiments, display 184 can also be combined with or serve as part of user interface 182 as a touch screen or touchless interface, for example.

Although not specifically identified in the figures, pumps 100A and 100B may include various alert components, such as speakers, displays or vibration components. These components may be independent features specifically designated as stand-alone alert components, or they may be components serving as alert components that share functionality with other pump components.

In general, infusion pumps 100A-B such as those disclosed are routinely operated by practitioners when in hospitals and in other medical care environments. Medical infusion pumps 100A and 100B are each examples of infusion pumps that can be suitable for use with embodiments discussed herein, though other pumps and devices can be used in various other embodiments of infusion systems utilizing subject matter hereof.

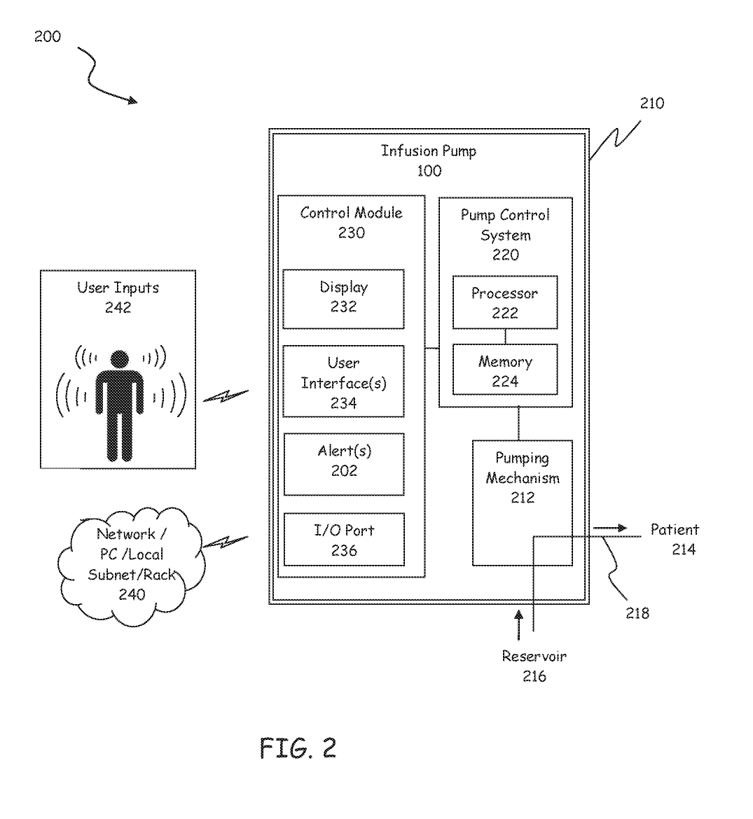

FIG. 2 is a block diagram of an infusion pump system 200 providing push alert notifications for practitioners. System 200 includes infusion pump 100 (that could be, for example, pump 100A or 100B of FIG. 1) incorporating an alert feature or features 202. Infusion pump 100 includes a pump housing 210 coupled with a pumping mechanism 212 that selectively delivers an infusate to a patient 214 from a reservoir 216 via an infusion line or tube 218.

In FIG. 2, infusion pump 100 includes a pump control system 220 with processor 222 and memory 224 programmable with selected protocols, profiles, segments of profiles, and other settings for controlling operation of pumping mechanism 212 such as, for example, the aforementioned syringe and ambulatory or peristaltic type mechanisms that can draw infusate from a reservoir 216 or other fluid or infusate source. In embodiments, reservoir 216 can comprise any suitable infusate supply, such as an IV bag, syringe, continuous supply, or other infusate storage device.

Processor 222 can be any programmable device that accepts digital data as input, is configured to process the input according to instructions or algorithms, and provides results as outputs. In an embodiment, processor 222 can be a central processing unit (CPU) configured to carry out the instructions of a computer program. Processor 222 can also or alternatively be an application specific integrated circuit (ASIC) or a field-programmable gate array (FPGA). Processor 222 is therefore configured to perform arithmetical, logical, and input/output operations.

In an embodiment, memory 224 can comprise volatile or non-volatile memory as required by the coupled processor 222 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example.

In FIG. 2, infusion pump 100 also includes a control module 230 for relaying commands to pump control system 220. Control module 230 includes a display 232 and at least one user interface 234. In some embodiments, user interface 234 works cohesively or cooperatively with display 232. In some cases, display 232 will be considered part of the user interface(s) 234. User interface 234 generally allows a user to enter various operating parameters, including but not limited to names, drug information, limits, delivery shapes, information relating to hospital facilities, as well as various user-specific operating parameters.

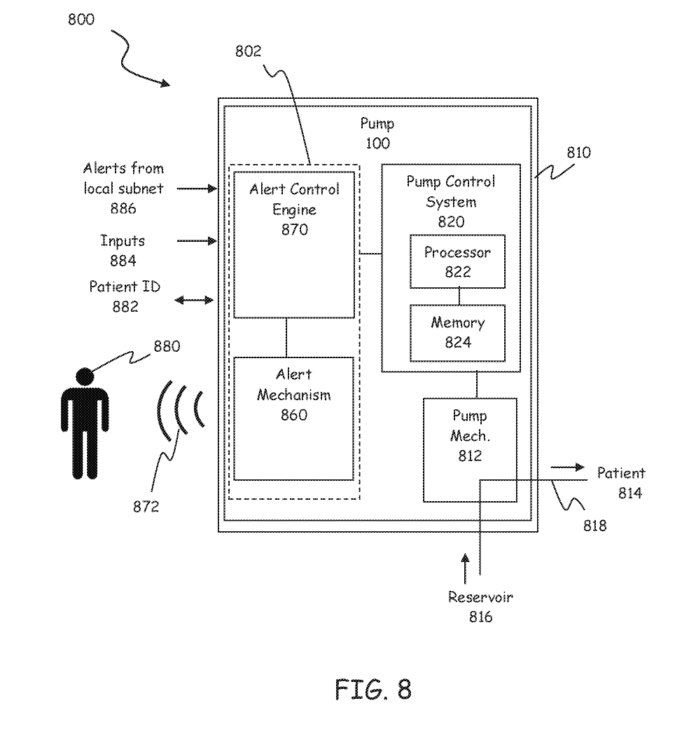

In FIG. 2, infusion pump 100 includes an alert feature or features 202 (collectively, “alert 202”) that make push alerts for infusion pump system 200 possible. Specifically, alert 202 may include features such as, individually or together, an alert component and an alert control engine. An alert component may include, individually or in various combinations with each other, a speaker, a visual display, a flashing light, a vibration component, or any other component or components that is or are capable of visual, audio, or tactile alarm display, broadcast, announcement or other warning/information dissemination to practitioners or other users. An alert control engine (not illustrated in FIG. 2) of alert 202 can serve to control and direct alert information and notifications in conjunction with pump control system 220. Additional information about these and other features of alert 202 are discussed in greater detail with respect to FIG. 8 and throughout the following disclosure.

For purposes of its use throughout this disclosure, the term “engine” can be defined as a real-world device, component, or arrangement of components implemented using hardware, or as a combination of hardware and software, such as by a microprocessor system and a set of particular program instructions that adapt or prompt the engine to implement the particular functionality, which (while being executed) transform a microprocessor system into a special-purpose device. A engine can also be implemented as a combination of the two, with certain functions facilitated by hardware alone, and other functions facilitated by a combination of software-controlled hardware. In certain implementations, at least a portion, and in some cases, all, of a engine can include the processor(s) of one or more computers that execute an operating system, system programs, and application programs, while also implementing the engine using multitasking, multithreading, distributed (e.g., cluster, peer-peer, cloud, etc.) processing where appropriate, or other such techniques. In addition, an engine can itself be composed of more than one sub-engines, each of which can be regarded as an engine, whether collectively or individually.

In some embodiments, infusion pump 100 of FIG. 2 includes a USB port or other appropriate input/output interface (I/O) port 236 for connecting infusion pump 100 to a network or computer 240 having software designed to interface with infusion pump 100. In some cases, I/O port 236 may also be useful for connecting to a rack for supporting pump 100 or a plurality of pumps 100, or other hardware, for localized networking or connectivity. Embodiments relating to rack and other subnet or hardware arrangements are contemplated as well. Power to infusion pump 100 can be accomplished via an AC power cord or internally provided battery.

User inputs 242 to the system are provided by programming of a user such as a nurse, physician, or other medical practitioner. User inputs 242 can include a variety of forms and be expressed in a variety of ways. User inputs 242 can be received by user interface 234, I/O port 236 or other practitioner input mechanisms.

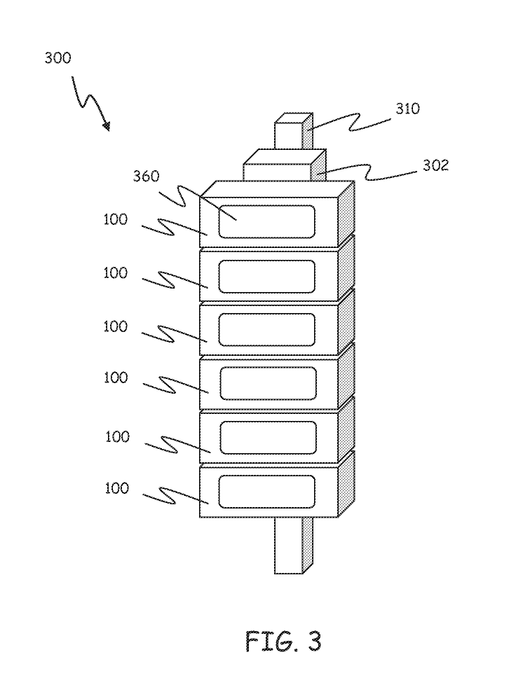

In FIG. 3, a plurality of infusion pumps 100 are depicted as each being installed in a generally vertical relationship along pole 310 as part of an alert notification system 300. Although illustrated fragmentally, it will be appreciated by those of skill in the art that pole 310 may be any suitable and generally vertical support structure such as is commonly used for suspending elevated intravenous (IV) fluid reservoirs, bags, and the like. A rack 302 is coupled to pole 310 by way of a suitable pole clamp (not illustrated) and pumps 100 are each removably couplable to rack 302. In other embodiments, rack 302 may be freestanding or coupled to other structures or support features. Accordingly, mounting rack 302 includes a plurality of infusion pumps 100 coupled to the mounting rack 302 that are vertically aligned and coupled to the mounting rack 302. Other arrangements are possible in embodiments as well. For example, pumps 100 could include mating features that enable pumps 100 to be physically connected to each other in, for example, a vertically-oriented stack of pumps 100. Infusion pumps 100 merely represent one type of medical device that could be mounted on a rack 302 or otherwise physically connected or stacked. Other medical devices of various shapes sizes and functions could be coupled to a rack as well or otherwise physically connected or stacked. Examples of other types of medical devices include patient vital sign monitors such as pulse oximeters, and other patient sensors and therapy devices such as blood rapid infusers, etc.

It is to be appreciated and understood generally that alert notification system 300 can be “localized” as parameters are configured and transmitted in close physical proximity and/or to a limited group of interconnected devices. In certain embodiments, communication is not performed in a system-wide, centralized manner but through local device to device interactions, via rack 302 or other intermediary structures in certain embodiments (such as in the aforementioned stacked or other physically connected embodiments) in which local transmission of information or operating parameters from one physical device to another is made possible.

In the example of FIG. 3, each of the pumps 100 can have a push alert component 360 individually or collectively as part of the rack 302. Alert components 360 can include speakers, visual displays, flashing lights, vibration components, and other components capable of visual, audio, or tactile alarm display, broadcast, announcement or other warning/information dissemination to practitioners or other users.

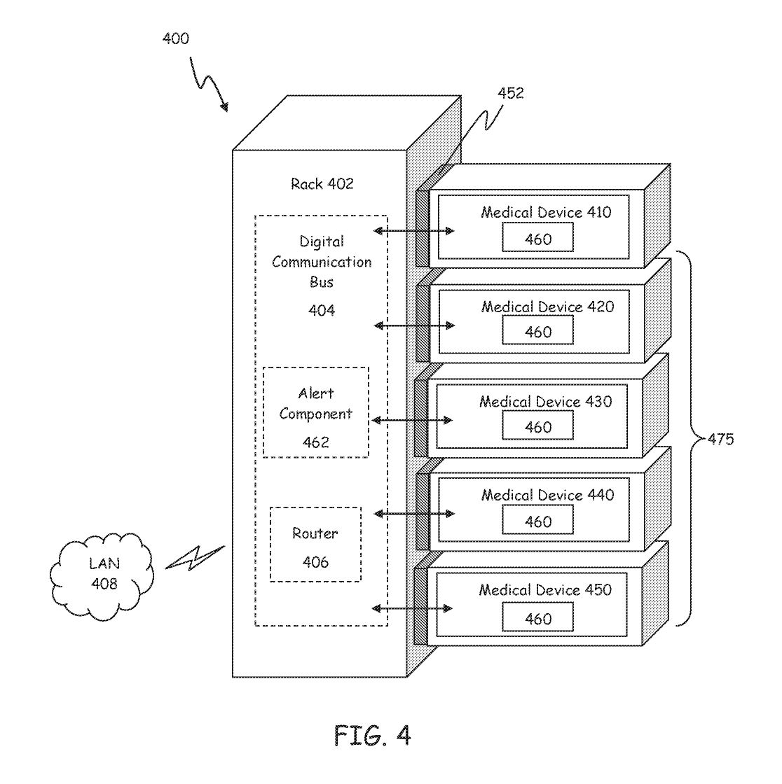

FIG. 4 depicts a diagram of a localized alert notification system 400, similar to the specific localized alert notification system 300 shown in FIG. 3. The system 400 includes a rack 402, digital communication bus 404, router 406, and a plurality of medical devices 410, 420, 430, 440 and 450. System 400 can, in certain embodiments, provide an arrangement in which infusion alerts and updates are distributed from one medical device to another or a plurality of other medical devices, all being coupled to a common local component or interface. The medical devices receiving infusion alerts and updates can generate appropriate audio, visual, or tactile alert notifications.

In certain embodiments, one medical device can be identified as a parent medical device such as, for example, device 410, and another or a plurality of other medical devices can be referred to as child medical devices such as, for example, 420, 430, 440 and 450. In some systems, any of the medical devices could be deemed the parent device as this function is not necessarily dictated by physical location or position. Further, while a plurality of potential child medical devices are shown in FIG. 4, this number of child medical devices should not be viewed as limiting. Fewer or additional medical devices may be present in certain embodiments.

In general, rack 402 can comprise various shapes and sizes. In some cases, rack 402 may be similar to rack 302 shown in FIG. 3. However, the shape and size of rack 402 is not limited to such an arrangement and can be a structure suitably adapted to effectively mount and connect the various medical devices desired. Rack 402 generally includes at least a plurality of mounting structures 452 configured to physically, communicatively, and removably couple, for example, the plurality of child medical devices 420, 430, 440 and 450 to a parent medical device 410. Mounting structures 452 can include any attachment structure or mechanism used to suitably couple the medical devices physically and communicatively to the rack 402.

Each of the medical devices may include an alarm component 460. Alarm components 460 can each generate audio, visual, or tactile alarms. In some embodiments, rack 402 can include a rack alarm component 462 as well. A rack alarm component 462 could be configured to provide an alert notification to a practitioner at any time when any of the medical devices coupled to rack 402 receives an infusion alert or update and can alarm in addition to alarm components 460 in the individual medical devices 410, 420, 430, 440 and 450. In other embodiments, rack alarm component 462 can be used to replace the individual alarm notifications from the individual medical devices to reduce alarm confusion and “alarm fatigue”.

Digital communication bus 404 is shown as integrated with or on rack 402. Digital communication bus 404 enables communication of operational information and programming between the medical devices. In some embodiments, this may be between the parent medical device 410 and the plurality of child medical devices 420, 430, 440 and 450 that are coupled to the rack 402. Medical devices 410, 420, 430, 440 and 450 may be infusion pumps as shown in, for example, FIG. 1A, FIG. 1B, FIG. 2, or FIG. 3, either individually or in various combinations with each other, in some embodiments.

In system 400 shown in FIG. 4, it is possible for parent medical device 410 to communicate alerts or infusion updates relevant to a particular patient in the plurality of child medical devices 420, 430, 440 and 450 by sending communications across or through digital communication bus 404 with appropriate instructions. Accordingly, an alert of the parent medical device will be provided to the child medical devices; and such alert can be communicated to a practitioner via alert components 460 and/or 462.

Digital communication bus 404 generally refers to a communication system that transfers data between components inside a computing device, or between computing devices. In some embodiments, bus 404 can include any or all related hardware components (wire, optical fiber, etc.) and software, including communication protocols. Bus 404 could utilize an Ethernet, SPI, CAN, RS232 or other communication architecture or method. In some embodiments, digital communication bus 404 may include a router 406. Router 406 can be configured to enable digital communications between medical devices 410, 420, 430, 440 and 450 that are physically and removably coupled to rack 402 and into a local area network (LAN) 408, in certain embodiments; and rack 402 could be, as aforementioned regarding rack 302, replaced with another arrangement of devices 410-450 such as, for example, provision of mating features that enable devices 410-450 to be physically connected to each other in, for example, a vertically-oriented stack. Although not specifically illustrated, in such a configuration stackable infusion pumps could be communicatively coupled by way of suitable electronic connections or other connectivity means between them and therefore be operative together to alert practitioners to changes required for the pumps as described herein but without use of a rack or rack-like component.

A rack 402 (or other arrangement) of medical devices 410, 420, 430, 440 and 450 can be an isolated subnet in a larger communication network. The medical devices on rack 402 can identify that only the medical devices also mounted to the same rack are viable partners or authorized for operation together. Such a configuration or association helps to prevent random medical devices in a facility from mistakenly receiving unnecessary alerts.

Using this system, it is possible to quickly and easily pass on notifications about infusion changes to practitioners. For example, when medical device 410 is an infusion pump, a change in an infusion order in or for device 410 can be passed to other medical devices 420, 430, 440 and 450 using a digital communication pathway through mounting rack 402. Changes in infusion orders can include new infusion orders, modifications to existing or pending infusion orders or other updates to a patient's treatment that potentially relate to that medical device.

Parent medical devices such as, for example, parent medical device 410 in FIG. 4, can be configured from a server, PC-based configuration tool, or in manufacturing, to be the “source” and have its infusion alert information passed to other child medical devices such as, for example, devices 420, 430, 440 and 450 in FIG. 4. For certain embodiments later discussed, these child medical device may be associated and collectively referred to as a group of selected devices 475 for another particular medical device 410 that takes the form of an infusion pump 100.

It is to be appreciated and understood that in some embodiments, the aforementioned parent-child arrangements of devices could also be configurable directly on designated parent devices (or any designated devices) themselves and not require external systems or protocols to establish such arrangements or relationships. It is also to be appreciated and understood that in some embodiments, the aforementioned parent-child arrangements of devices could also be configurable according and responsive to physical arrangements of the devices that have been stacked and/or racked. For example, in such a particular embodiment, a topmost or highest pump in a vertically-oriented stack or rack of pumps and other devices could be designated as the parent according to its physical position therein; and in another such embodiment, a bottom or lowest pump in a vertically-oriented stack or rack of pumps and other devices could be designated as the parent.

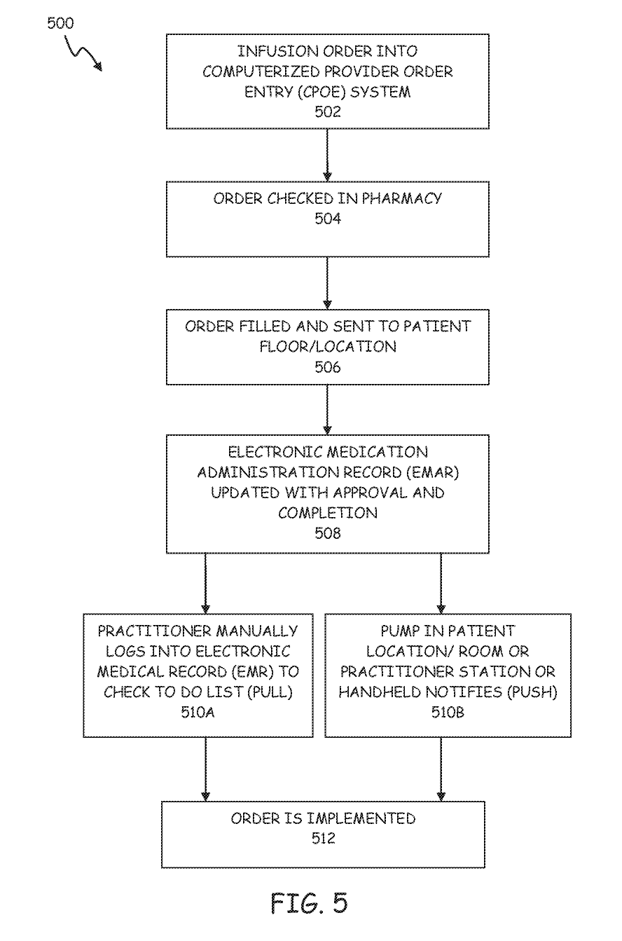

FIG. 5 shows a method 500 of providing alerts via infusion pumps. This method indicates both a push alert option and a pull alert option by which a practitioner could be informed of infusion order changes, additions or updates. As shown in FIG. 5, at 502 an infusion order is received for a patient in a computerized physician order entry (CPOE) system. The infusion order includes a patient identifier. An infusion order may include new orders, updates, etc. For compliance with healthcare privacy laws and regulations in the United States and elsewhere, a patient identifier would not need to be displayed, but can include some indication uniquely and specifically tied to a patient that is readily recognizable by various devices and systems.

Next, at 504, the infusion order is checked into a pharmacy. This action can include obtaining verification of the infusion order from the pharmacy, for example. The method further can include filling the infusion order and sending it to the patient floor or location at 506. More generally, this step includes sending the infusate requested in the infusion order from the pharmacy to a patient location. At 508, the electronic medication administration record (EMAR) system is updated with an approval to complete or implement the infusion order. This may include an indication of an approval to administer the order and an indication of completed filling or implementation of the order by the pharmacy and/or request for delivery or delivery of the infusate to a patient location. As in past systems and methods, the practitioner can manually log into the electronic medical record (EMR) to check a “to do” list, as shown in 510A. In effect, a practitioner is able to inquire to “pull” this to do list from the system. In addition to this, the disclosed method also includes providing a “push” notification, as indicated at 510B. Such a “push” notification is provided from an alert component in one or more devices associated with the patient identifier of updates to the EMAR system.

As referenced in other locations, an alert component may include a variety of components capable of visually, audibly or tactically providing warnings or alert information. This notification does not require an inquiry initiated by a practitioner into an EMAR system application or otherwise opening or interacting with such an application by the practitioner. In various embodiments, the notification is provided by at least an infusion pump at the patient location or otherwise co-located with the patient, such as in a room with the patient. Other devices associated with the patient identifier providing a notification from an alert component can include additional infusion pumps proximate the patient, practitioner workstations for particular units or care areas, practitioner handheld devices, and other patient monitoring, charting, and diagnostic equipment, for example. Following notification, the practitioner then administers the ordered infusate to the patient and, accordingly, completes or implements the infusion order as indicated at 512.

A push notification, as at 510B, can also further utilize one or more alert escalation processes in some embodiments. For example, an alert escalation process can begin with generally modest initial alerts delivered by one or more visual, audible, or tactile notifications and escalate into increasingly conspicuous and difficult-to-overlook alerts. If the order or alert is not fulfilled or addressed within a predetermined amount of time, further or more concentrated alerts may be issued. These further alerts may increase one or more of: frequency, volume, brightness, degree of tactile movement, or other attention-getting characteristics. Alerts can be designed to gradually or quickly increase. This type of escalation process could be implemented in or adapted to any of the embodiments discussed throughout this disclosure. Such an escalation process is made possible by the “push” nature of the notifications and can be appropriately tailored to the urgency of the situations or types of notifications in certain embodiments.

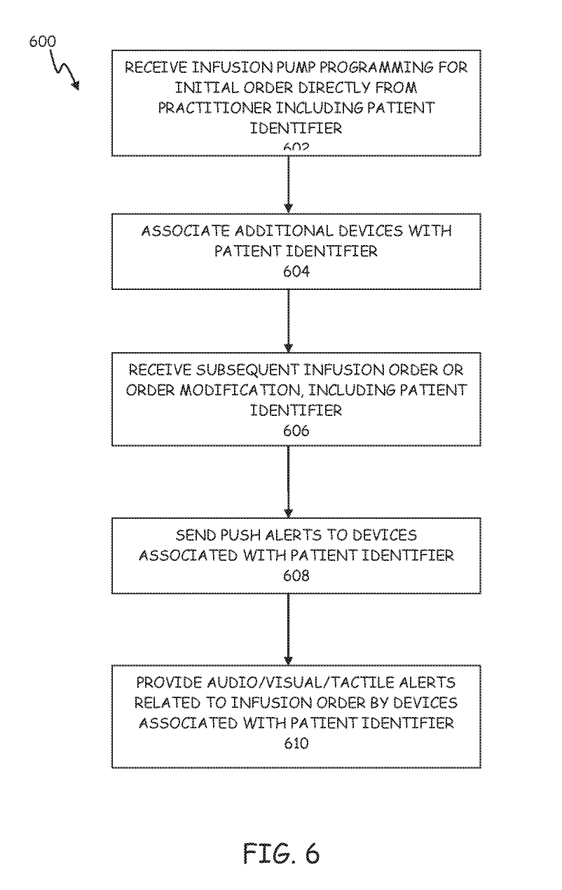

FIG. 6 shows a method 600 of providing push alerts of infusion pump orders. The method includes receiving manual or local programming by a practitioner of a first infusion pump order, including a patient identifier of a patient, as indicated at 602. Infusion pump orders may include new orders, updates, etc. This order or programming could be entered directly into the user interface of an infusion pump by a practitioner, for example. The method also includes, associating a plurality of additional devices with the patient identifier, as indicated at 604. This association permits, for example, future infusion orders, auto-programming messages, and work orders to be sent to these devices. Unused pump may be some of the plurality of additional devices that could be associated with a patient identifier. A second (subsequent) infusion order is received, at 606, in which is associated with the patient identifier. This second infusion order may be received in various ways. In some cases the second infusion order will be received via the infusion pump connection to the local subnet, via a hospital-wide patient management system, or directly from a practitioner by manual programming. Receipt of the second infusion order or any subsequent orders is followed by sending one or more push alerts, as indicated at 608. This includes sending a message to the plurality of devices associated with the patient identifier regarding the second infusion pump order. The plurality of devices respond by providing push alert notifications. Specifically, one or more audio, visual, or tactile alert notifications are provided regarding the second infusion pump order from an alert component of each of the plurality of devices associated with the patient identifier without requiring a practitioner action (such as a practitioner-initiated inquiry into the EMAR system), as shown at 610. Accordingly, because practitioners are provided alerts in real time without relying on practitioner-initiated, periodic checking of (or, “pulling” from) the EMAR system, efficiency of care is enhanced.

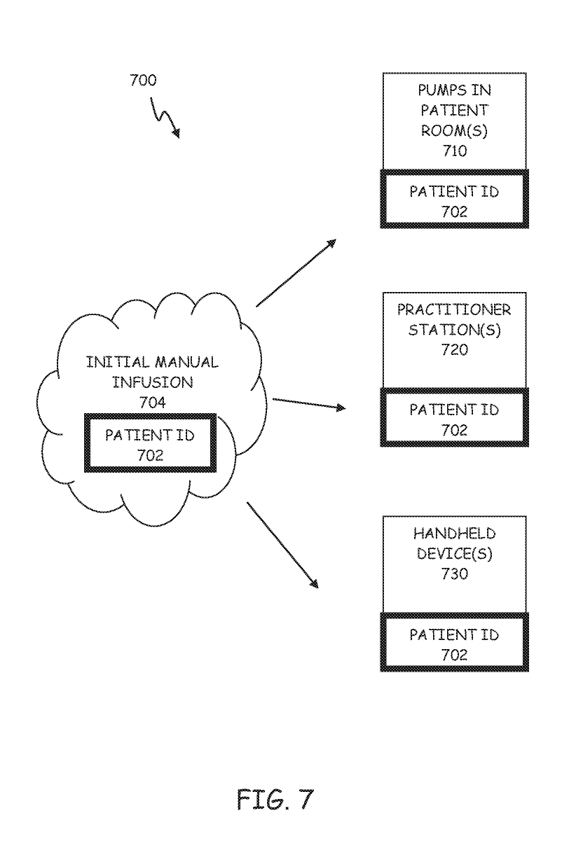

FIG. 7 depicts a system 700 in which patient identifiers 702 are associated with various devices in accordance with the novel and inventive subject matter hereof. The diagram of the embodiment shown, includes an initial manual infusion 704 command to one of the infusion pumps in a medical care facility. In the programming of this initial infusion 704, a patient identifier 702 for the patient is associated with the pump. Once this initial infusion is programmed, the patient identifier 702 is communicated to all other devices in the local network associated with that particular patient. Devices associated with the patient identifier 702 can include pumps in patient room(s) 710, practitioner workstation(s) 720 for a particular unit or care area, and handheld device(s) 730 for practitioners treating a particular patient. Patient identifiers 702 need not be displayed on the device. In some cases, patient identifiers 702 will be tied to a local subnet or rack subnet.

FIG. 8 shows a diagram of an infusion pump system 800 with push alert notification in accordance with the novel and inventive subject matter hereof. The system includes an infusion pump 100 having a number of alert features 802. The system 800 should be understood in conjunction with the system 200 of FIG. 2, but with more specific disclosure related to embodiments of the alert features of the infusion pump 100. Embodiments contemplated include both the arrangements of FIG. 2 and FIG. 8 as well as various combinations of the features disclosed and understood from these disclosures. Infusion pump 100 includes a pump housing 810 coupled to a pumping mechanism 812 that selectively delivers an infusate to a patient 814 from a reservoir 816 via an infusion line or tube 818. The infusion pump 100 also includes a pump control system 820 including a processor 822 and a memory 824 programmable to control operation of the pumping mechanism 812. It is to be appreciated and understood that more specific information about these and other similar corresponding components of infusion pump 100 can be ascertained from inspection of the example of FIG. 2 and the foregoing descriptions thereof.

The infusion pump 100 also includes alert features 802 including an alert component 860 and an alert control engine 870. Alert component 860 is coupled with the pump housing 810 and provides audio, visual, or tactile alerts 872 to a practitioner 880. Further, alert control engine 870 is in operable communication with the pump control system 820. The alert control engine 870 includes programming that associates a patient identifier 882 for the patient 814 with the infusion pump 100. Pump configuration inputs 884 can be received directly from a practitioner/user 880 or from a local subnet. Pump configuration inputs 884 may include an association of a patient identifier 882 during the initial manual programming by the practitioner/user 880. The alert control engine 870 is configured to receive alert messages 886 from a local subnet related to infusion orders associated with the patient identifier 882. The alert control engine 870 is also programmed to instruct the alert component(s) 860 to provide one or more alerts 872 to a practitioner/user 880 related to infusion orders without requiring user inquiry or action.

In FIG. 8, the alert features are shown as being operatively coupled with one another and to the pump control system 820, although other configurations are deemed possible and taught by this disclosure as well. In certain embodiments, the alert control engine 870 and alert component 860 will be part of a control module as shown in FIG. 2. In other embodiments, components will operate outside the control module or directly within the pump control system. Further, alert component 860 and the alert control engine 870 may share components with other modules or systems shown. For example, display 232 in FIG. 2 may act as alert component 860 of FIG. 8 in some embodiments or the alert control engine 870 may share or utilize the processor and memory of the pump control system. Irrespective of a particular embodiment, it is to be appreciated and understood that neither the block diagram of the infusion pump 100 shown in FIG. 2 or FIG. 8 is comprehensive, exhaustive, or exclusive with respect to the novel and inventive subject matter hereof.

The infusion pump 800 shown in FIG. 8 may be understood to be part of a larger system of providing push alert notifications for infusion pumps. In some embodiments the system could be part of a local subnet, as understood from FIGS. 3 and 4 for example. Accordingly, in some embodiments, the system can include a group of selected devices 475 such as medical devices (for example, medical devices 420, 430, 440, and 450), associated with a local subnet, as shown in FIG. 4, for example. The system can also include an additional medical device 410, such as, an infusion pump 100, shown in FIG. 8, for example. Infusion pump 100 may be part of and associated with the local subnet as well. The infusion pump 100, including a pump housing 810, can be coupled to a pumping mechanism 812. Pumping mechanism 812 is adapted to selectively deliver infusate to a patient 810. The infusion pump 100 may also include a pump control system 820 including a processor 822 and a memory 824 programmable to control operation of the pumping mechanism 812.

The infusion pump 100 also includes an alert control engine 870 that is in operable communication with the pump control system. The alert control engine 870 includes programming that associates a patient identifier, for sending future notifications. The patient identifier can be associated with the group of selected devices 475 following receipt of programming for a first order for the infusion pump for the patient. The alert control engine 870 also includes programming for future updates. Specifically, the alert control engine 870 receives a second order for the infusion pump, generates an alert message related to the second order, and sends the alert message to the group of selected devices 475. Each device of the group of selected devices 475, and the infusion pump, can each contain an alert component 460 that provides an audio alert, a visual alert, or a tactile alert 872 when the alert message is sent.

Accordingly, based on the foregoing disclosure, pumps or medical devices in a patient room or otherwise co-located with the patient are able to notify a practitioner of an impending pump order. Pumps and/or devices are associated with the patient to advantageously push alerts to appropriate locations or care areas of a hospital and to appropriate practitioners and their electronic devices. Local subnets make associations to patient rooms and locations possible once an initial infusion is started and automatically sent through programming protocols including the patient identifier. The disclosed systems and pumps are particularly useful in medical care facilities where unused pumps and other medical devices may be located in patient rooms. By way of the novel and inventive devices, systems, and methods providing push alerts to infusion pumps and related devices hereof—as described by example or otherwise contemplated herein—it is to be appreciated and understood that such unused pumps and devices can be effectively utilized to issue relevant notifications to practitioners. In certain embodiments, racks like those described with reference herein to FIGS. 3 and 4 could be permanently mounted in patient rooms and pumps and other devices attached to those racks could be located based upon the rack location.

It is also to be appreciated and understood that the novel and inventive devices, systems, and methods providing push alerts to infusion pumps and related devices hereof—as described by example or otherwise contemplated herein—could also be advantageously configured and employed for a plurality of pumps for a primary infusion and a secondary infusion, sometimes known in the art as “piggybacking.” For example, a first pump could be configured and commanded to administer a first infusate to a patient through a fluid line or tubing while a second pump could be configured and commanded to administer a second infusate along the same line or tubing to the patient such that the second infusate is provided concurrently or consecutively in “piggybacked” fashion with the first infusate. Such a “piggyback” system is also described in U.S. provisional patent application Ser. No. 62/158,213 filed on 7 May 2015, titled “Server Systems for Coordinating and Controlling Infusion Pumps”, with the disclosure thereof being incorporated herein by reference thereto. A copy of which is attached as an Appendix to the present application. In particular in such an embodiment having a piggyback capability, for example, an order for a secondary infusion could be pushed to a parent pump as a “piggyback alert”. Then, upon receiving and acknowledging the piggyback alert, an infusion therapy in progress could be interrupted on a syringe pump, for example, to provide a secondary infusion from an LVP via the same tubing; and when the piggybacked LVP infusion is complete, the original syringe pump infusion could be resumed.

It should also be appreciated that the exemplary embodiment or exemplary embodiments are only examples, and are not intended to limit the scope, applicability, or configuration of the novel and inventive subject matter hereof in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the subject matter hereof as set forth in the appended claims and the legal equivalents thereof. For example, although described and depicted as being in generally vertical stacks and racks, pumps and devices—as described by example or otherwise contemplated herein—could be provided in any other suitable orientations or arrangements in a particular embodiment of novel and inventive subject matter hereof. For example, although pumps 100 are depicted as being arranged vertically in FIG. 3, it is to be appreciated and understood that they could instead be arranged horizontally and physically supported and communicatively connected horizontally by way of any suitable horizontal stacking (or side-by-side coupling) or racking components and techniques.

Embodiments described by example or otherwise contemplated herein are intended to be illustrative and not limiting. Additional embodiments are within the claims. Although subject matter hereof has been described with reference to particular embodiments, workers skilled in the art will recognize that changes may be made in form and detail without departing from the spirit and scope of the subject matter. For example, in addition to the “pushing” out of an infusion order change or alert to a change needed or desired for a particular infusion therapy being delivered to a patient, a particular device, system, and method hereof that provides a push alert to an infusion pump and related devices could be configured to push out a recommended cancellation of an active infusion therapy, in progress, but subject in most such embodiments to a required confirmation from the practitioner before the cancellation is imposed and the infusion therapy is stopped.

Also, it is to be appreciated and understood that although not specifically illustrated in the drawings, alert components as have been described by example or otherwise contemplated could include or be, individually or in various combinations with each other, a kinetic/motion-based device or system such as, for example, a suitably sized small waving flag (whether physical and mechanical, or virtual and electronic). In an embodiment, a kinetic/motion-based device or system could include at least one motion sensor so that the alert component would be effective if it would inadvertently not be visually observed by the practitioner. In an embodiment, a kinetic/motion-based device or system could comprise an infrared (IR) detector system. Such a device or system could, for example, also be configured to be recognizable on a security or patient monitoring camera but not in the patient's room environment, to minimize unwanted sensory stimuli or distraction to the patient.

Various modifications to subject matter hereof may be apparent to one of skill in the art upon reading this disclosure. For example, persons of ordinary skill in the relevant art will recognize that the various features described for the different embodiments of the subject matter hereof can be suitably combined, un-combined, and re-combined with other features, alone, or in different combinations, within the spirit of the subject matter. Likewise, the various features described above should all be regarded as example embodiments, rather than limitations to the scope or spirit of the subject matter. Therefore, the above is not contemplated to limit the scope of the subject matter.

For purposes of interpreting the claims for subject matter hereof, it is expressly intended that the provisions of 35 U.S.C. § 112(f) are not to be invoked unless the specific terms “means for” or “step for” are recited in a claim.

1. A method of providing push alerts via infusion pumps, comprising:
receiving an infusion order for a patient in a computerized physician order entry system including a patient identifier;
obtaining verification of the infusion order from a pharmacy;
sending an infusate requested in the infusion order from the pharmacy to a patient location;
updating an electronic medication administration record (EMAR) system with an approval to implement the infusion order;
providing a notification from an alert component in one or more devices associated with the patient identifier of updates to the EMAR system, without a practitioner initiated inquiry, wherein at least one of the one or more devices is an infusion pump at the patient location.

2. The method of providing push alerts of claim 1, wherein at least one of the one or more devices includes a practitioner workstation.

3. The method of providing push alerts of claim 2, wherein at least one of the one or more devices includes a practitioner handheld device.

4. A method of providing push alerts of infusion pump orders, comprising:
receiving manual programming by a practitioner of a first infusion pump order, including a patient identifier of a patient;
associating a plurality of devices with the patient identifier;
receiving a second infusion pump order associated with the patient identifier;
sending a message to the plurality of devices associated with the patient identifier regarding the second infusion pump order; and
providing one or more audio, visual, or tactile alert notifications regarding the second infusion pump order from an alert component of each of the plurality of devices associated with the patient identifier without requiring a practitioner action.

5. The method of claim 4, wherein the plurality of devices include an infusion pump, a practitioner workstation, and a practitioner handheld device.

6. The method of claim 4, wherein the plurality of devices are co-located with the patient.

7. An infusion pump with push alert notification, comprising:
a pump housing;
a pumping mechanism coupled to the pump housing that selectively delivers an infusate to a patient;
a pump control system including a processor and a memory programmable to control operation of the pumping mechanism;
an alert component, coupled with the pump housing, providing audio, visual, or tactile alerts; and
an alert control engine, in operable communication with the pump control system, including programming that:
associates a patient identifier for the patient with the infusion pump;
receives an alert message from a local subnet related to an infusion order associated with the patient identifier;
instructs the alert component to provide an alert notification to a practitioner related to the infusion order without requiring an inquiry by the practitioner.

8. The infusion pump of claim 7, wherein the alert component provides an audible message.

9. The infusion pump of claim 7, wherein the alert component provides a visually displayed alert.

10.-16. (canceled)
